(19) United States
(12) Patent Application Publication
(10) Pub. No.: US 2011/0112356 A1
(43) Pub. Date: May 12, 2011

(54) Dementia treatment

(76) Inventor: Mark Ellery Ogram, Tucson, AZ (US)

(21) Appl. No.: 12/590,646

(22) Filed: Nov. 12, 2009

(51) Int. Cl. A61M 21/00; G08B 1/08

(52) U.S. Cl. 600/27; 340/539.11

(57) ABSTRACT

A memory treatment mechanism which utilizes subliminal messages communicated via a display. The subliminal messages contain patient specific information such as the patient's name, their relatives' pictures and names, where they lived, etc. Using a remote sensor/switch, the personal subliminal information is curtailed when a non-patient is in the room and could be exposed to the personal subliminal information. In a care facility, a single computer directs these patient related subliminal messages to a number of displays.

### BACKGROUND OF THE INVENTION

This invention relates generally to the treatment of mental conditions and more particularly to the treatment of dementia and Alzheimer.

Perhaps the cruelest affliction that patients and their families endure is that of dementia or Alzheimer's disease. While the patient retains their functioning memory, the underlying knowledge is lost. The patient then loses their ability to recognize faces, understand relationships, and appreciate where they are. Confusion reigns.

While these afflictions are extremely difficult for the patient, there is a pronounce affect on the family and friends of the patients. A grandchild can carry the hurt from not being recognized by a grandparent for years or decades.

For this reason, there has been a significant effort to identify when dementia or Alzheimer's is present. Examples of such diagnostic efforts are: U.S. Pat. No. 7,598,049, entitled “Methods for Diagnosis of Alzheimer's Disease in Blood Samples” issued to Ray, et al. on Oct. 6, 2009; U.S. Pat. No. 7,577,472, entitled “MRI Method for Producing an Index Indicative of Brain Disorders” issued to Li, et al. on Aug. 18, 2009; and, U.S. Pat. No. 7,575,876, entitled “Biomarkers for Neurodegenerative Disorders” issued to Zhang on Aug. 18, 2009; all of which are incorporated hereinto by reference.

While there have been great strides in medicating the patient, these treatments have not been successful to the extent that is required. Such treatments include: U.S. Pat. No. 7,601,740, entitled “Selective Serotonin 2A/2C Receptor Inverse Agonists as Therapeutics for Neurodegenerative Diseases” issued to Weiner, et al. On Oct. 13, 2009.

When the diseases progress, the caregivers are left with few treatment options and are forced to take steps to protect the patient. Some such protections are described in U.S. Pat. No. 7,573,371, entitled “Alarm System for Dementia Patients” issued to Miller on Aug. 11, 2009; incorporated hereinto by reference.

There has been a great deal of treatment modalities for dementia, but these have not been very successful and often require intensive caregiver interaction. Such modalities include: U.S. Pat. No. 7,563,808, entitled “Methods for Treating Cognitive Impairments or Dementia” issued to Pratt on Jul. 21, 2009; U.S. Pat. No. 7,557,109, entitled “Treatment for Attention-Deficit Hyperactivity Disorder” issued to Brunner, et al. on Jul. 7, 2009; U.S. Pat. No. 7,551,957, entitled “Electromagnetic Therapy Device and Methods” issued to Whelan, et al. on Jun. 23, 2009; and, U.S. Pat. No. 7,540,615, entitled “Cognitive Training Using Guided Eye Movements” issued to Merzenich, et al. on Jun. 2, 2009; all of which are incorporated hereinto by reference.

It is clear from the foregoing that there is a significant need for mechanisms which will assist the patient resist the assault from dementia and Alzheimer.

### SUMMARY OF THE INVENTION

The invention is a memory treatment mechanism which utilizes subliminal messages communicated via a display.

The monitor/display used in this invention is also well known to those of ordinary skill in the art and include: U.S. Pat. No. 7,545,952, entitled “Image or Video Display Devices” issued to Brundage, et al. on Jun. 9, 2009; and, U.S. Pat. No. 7,545,952, entitled “Image or Video Display Devices” issued to Brundage et al. on Jun. 9, 2009; both of which are incorporated hereinto by reference. The preferred display is television which delivers entertainment content to the viewer or patient.

Intermixed with the entertainment content, are subliminal messages. Subliminal messages are below the threshold of responsiveness or consciousness. The viewer is not aware of the messages but these same messages register on the brain without any conscious activity on the viewer's part.

Subliminal messaging is well known in the art and includes such techniques described in: U.S. Pat. No. 7,602,443, entitled “Methods for Improved Modulation of Video Signals” issued to Reynolds, et al. on Oct. 13, 2009; U.S. Pat. No. 7,567,686, entitled “Hiding and Detecting Messages in Media Signals” issued to Rhoads on Jul. 28, 2009; U.S. Pat. No. 7,460,667, entitled “Digital Hidden Data Transport (DHDT)” issued to Lee, et al. on Dec. 2, 2008; and U.S. Pat. No. 7,532,725, entitled “Systems and Methods for Permitting Open Access to Data Objects and for Securing Data Within the Data Objects” issued to Moskowitz et al. on May 12, 2009; all of which are incorporated hereinto by reference.

The subliminal messages of the invention contain two different content types: general/generic information; and patient specific information.

The general information is such items as the date (“Monday, Oct. 19, 2009”), where they are located (“St. Mary's Long Care Facility in Tucson, Ariz.”), the president's name (“The President is Borak Obama”), and other such information. This information is intended to keep the patient aware of their current situation.

The patient specific subliminal messages contain information unique to the patient such as the patient's name (“Your name is John Smith”), their relatives' pictures and names (“This is your wife”), where they use to live (“You used to live here in Phoenix” together with a picture of the house), etc. This information is intended to keep the patient aware of their history by providing unconscious reminders.

Another type of patient specific subliminal messages contain information about current events happening with the patient. These might be such items as: special events (“Congratulations, today is your birthday, February 3”), planned activities (“today you will get physical therapy on your shoulder”), reminders of things to do (“remember to walk a little today”), their surroundings (“the bathroom is on your right”), and other pertinent information.

Ideally, the subliminal messages contain not only textual information, but visual information as well. Visual information in the form of photos and such are extremely helpful for the patient in recognizing family and places.

Being exposed to the patient specific subliminal messages can be disquieting to anyone other than the patient. For this reason, it is ideal to curtail transmission of the patient specific message when a non-patient is in the room. Using a remote sensor/switch, the personal subliminal information is curtailed when a non-patient is in the room and could be exposed to the personal subliminal information.

While a manual switch at the door is one mechanism which can be used by a care-giver to suspend the transmission of personal information, in another embodiment a presence sensor is used to automatically suspend the personal information.

Those of ordinary skill in the art readily recognize a variety of mechanisms which will serve this purpose including, but not limited to: U.S. Pat. No. 7,570,152, entitled “Method and Apparatus for Temporarily Disabling a Patient Monitor” issued to Smith, et al. on Aug. 4, 2009; incorporated hereinto by reference.

In a care facility, a single computer directs these patient related subliminal messages individually to a number of displays or televisions. This central location permits a more uniform distribution and control of the subliminal messages.

Another use of the subliminal messages applies not just for the patient but also for the care-giver. When the care-giver's presence in the room is sensed, queries or instructions can be sent through the monitor in the patient's room to assist the care-giver in providing proper care. These types of care-giver subliminal messages might be: patient care (“Remember to turn the patient regularly”), preparatory type of information (“Mr. Smith is going to have visitors, comb his hair and wash his face”), cautionary (“The patient is extremely allergic to peanuts”), and other type of information.

Ideally, the care-giver subliminal messages are also sent via displays to the nursing station to keep the staff cognizant of the daily duties.

Through the use of the various types of subliminal messages, the patient's slide into dementia is resisted and the patient receives better and consistent care.

The invention, together with various embodiments thereof will be more fully explained by the accompanying drawings and the following description thereof.

### DRAWINGS IN BRIEF

FIG. 1 is diagram illustrating a multi-user embodiment of the invention.

FIGS. 2A, 2B, and 2C illustrate specimen subliminal messages for patient specific information, generic type information, and care-giver information.

FIG. 3 is a flow chart of an embodiment of the operation of the invention.

FIG. 4 is a diagram illustrating the preferred switch used to identify when a non-patient is in the room.

### DRAWINGS IN DETAIL

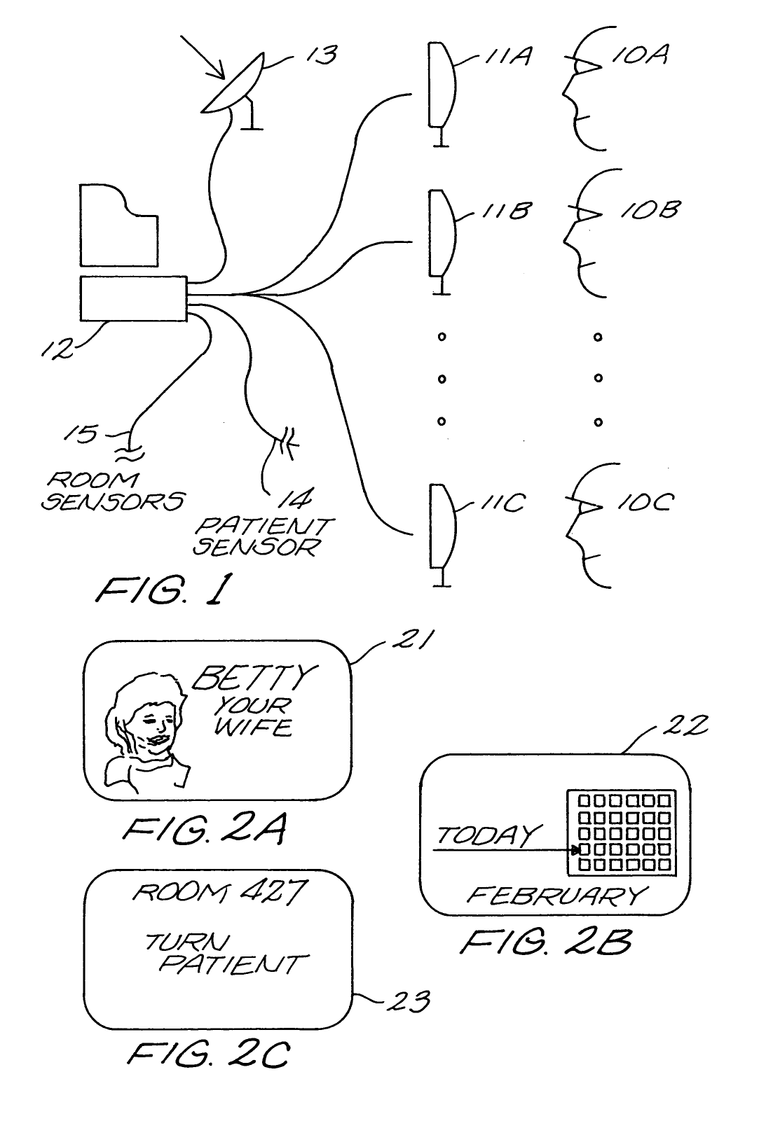

FIG. 1 is diagram illustrating a multi-user embodiment of the invention. Computer 12 controls the dissemination of visuals to the various displays 11A, 11B, through 11C. Each display has its viewer 10A, 10B, through 10C respectively.

The visuals used by computer 12 is collected from antenna 13 (receiving television programming) and patient sensors 14. The visual information from these two sites is then mixed with the appropriate subliminal information and sent to the proper display.

As example, for viewer 10A, a patient, the television information from antenna 13 has subliminal messages of both generic nature (e.g. the date) together with personal data (e.g. the name of the spouse) for viewer 10A. This information is communicated to display 11A until such time that the room sensor 15 indicates that another person is also able to see display 11A, at that time, computer 12 curtails the personal data subliminal messages until the room sensor 15 indicates that viewer 10A is alone.

Another example is when computer 12 communicates patient information from patient sensor 14 to display 11C which is now monitored by care giver/viewer 10C. The information from patient sensor 14 includes any number of patient specific information such as blood pressure, pulse rate, body temperature, etc. Computer 12 intermixes subliminal messages appropriate for the care giver/viewer 10C (i.e. “Remember to Medicate Room 132A”) into this stream of information to provide a subliminal reminder of the care giver's duty.

This later example is also useful for non-medical applications where informational screens are routinely monitored by personnel. This would include power plant operations, security guard stations, and phone operators. In these cases, the subliminal messages are used to provide reminders to the user/viewer (e.g. “Closely monitor the cooling status”; “Room 132 is supposed to be empty”; and, “Be courteous”).

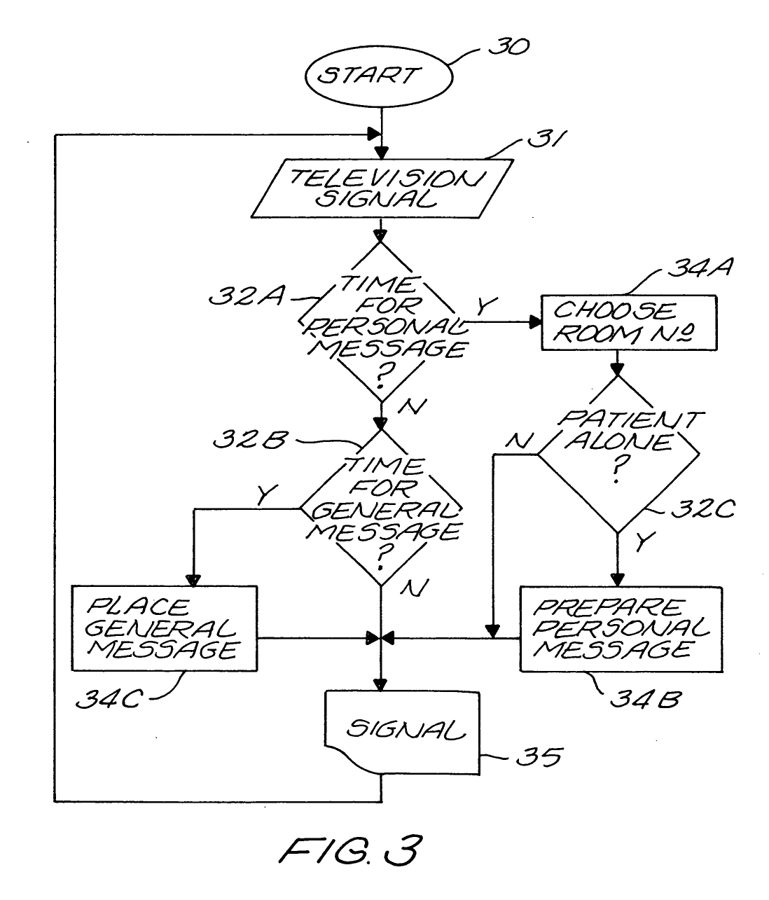

FIGS. 2A, 2B, and 2C illustrate specimen subliminal messages for patient specific information, generic type information, and care-giver information.

Referring to FIG. 2A, display 21 is used as a subliminal message to communicate personal information to the user/viewer. This illustration shows a picture of the viewer's wife together with the wife's name and identifies the individual as their wife. This subliminal reminder assists the patient in fighting dementia with a reminder so that when the wife enters the room, the patient remembers her, thereby both reducing the confusion to the patient and anxiety to the wife.

FIG. 2B shows an example of a subliminal display 22 which assists with generic information. The date is shown to the patient to keep the patient current. Other examples of generic information might be location (e.g. “You are in Phoenix, Ariz.), current events (e.g. “The president is Borak Obama”), and many others. These generic messages are ideally tailored to help “fill in” gaps which are developing in the patient's memory.

A subliminal message for the care giver is shown in FIG. 2C. Here, the message “Room 427, turn patient” is shown on display 23 to remind the care giver of a task at hand. Other messages might be “Meds are due to John Smith”, or “Mrs. Smith is subject to falls, watch her closely”.

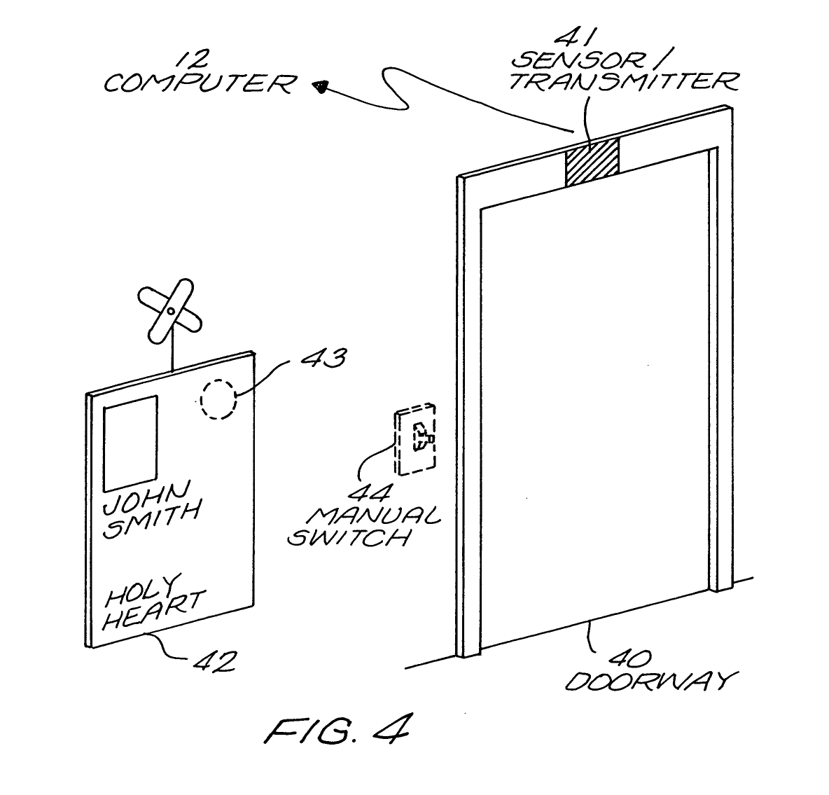

FIG. 3 is a flow chart of an embodiment of the operation of the invention used for a patient's room. Once the program starts, the television signal is obtained 31. A determination 32A is made if it is time for a personal message; if it is (Y), then a room number is chosen 34A and a determination is made if the patient is alone 32C. If the patient is alone (Y), then the personal message is prepared and placed in the television content 34B and the combined signal is communicated to the room 35, and the program returns to gather to television signal 31. Should the patient not be alone when a determination is made 32C (N), then the personal message is not sent.

Should the timing not be appropriate for a personal message (#32A (N)), then a determination is made on if a generic message's time is appropriate 32B. If it is (Y), then the generic message is placed in the television signal 34C and the combined message is communicated 35 to the patient.

If the time is not appropriate (N) for a generic message 32B, then the television signal without the generic subliminal message is send to the patient 35.

In this manner, appropriate subliminal messages are communicated to the patient within the television signal.

FIG. 4 is a diagram illustrating the preferred switch used to identify when a non-patient is in the room. In this embodiment, doorway 40 has a sensor 41 placed thereon. Sensor 41 communicates a signal to computer 12 to curtail the transmission of personal data as outlined above.

Sensor 41 is responsive to an embedded chip 43 which is placed within a care giver's identification badge 42. When the care giver passes through doorway 40 (going into the room), sensor 41 is tripped by chip 43 and computer 12 is notified that the patient is not alone in the room; when the care giver returns through doorway 40, again sensor 41 is tripped by chip 43 and computer 12 is notified that the patient is now alone in the room and the transmission of personal information is permitted.

In this way, the care giver not exposed to confusing information (personal information about the patient's history, not the care giver's own) while treating the patient.

Another embodiment for notifying computer 12 is a simple switch 44 which is manually closed or opened by the care giver when entering or leaving the room.

It is clear that the present invention provides for a mechanism which assists the patient in maintaining their memories and in resisting the demise from dementia and Alzheimers disease.

What is claimed is:

1. A memory treatment mechanism comprising:
a) a display communicating entertainment information to a patient;
b) a control mechanism, communicating electronic signals with said display, said control mechanism periodically interspersing subliminal content containing patient-specific messages into said entertainment information.

2. The memory treatment mechanism according to claim 1, wherein said subliminal content further includes generic messages.

3. The memory treatment mechanism according to claim 2,
a) further including a memory obtains said patient-specific messages and said generic messages; and,
b) wherein said control mechanism accesses said patient-specific messages and said generic messages from said memory.

4. The memory treatment mechanism according to claim 3,
a) further including a switch selectively producing a signal; and,
b) wherein said control mechanism suspends patient-specific messages from said subliminal content in response to the signal from said switch.

5. The memory treatment mechanism according to claim 4:
a) further including:
1) an electronic identifier worn by a human, and,
2) a sensor responsive to said electronic identifier and generating a presence signal when said electronic sensor is in a room with said display; and,
b) wherein said switch is responsive to said presence signal.

6. The memory treatment mechanism according to claim 5, wherein:
a) said electronic identifier is worn by the patient; and,
b) wherein said switch is activated when said electronic identifier is not within a room with said display.

7. The memory treatment mechanism according to claim 5,
a) wherein said electronic identifier is worn by a care-giver; and,
b) wherein said switch is activated when said electronic identifier is within a room with said display.

8. A memory treatment mechanism comprising:
a) a television communicating an entertainment program to a patient;
b) a controller periodically interspersing subliminal content containing patient-specific messages and generic messages into said entertainment program.

9. The memory treatment mechanism according to claim 8,
a) further including a memory containing said patient-specific messages and said generic messages; and,
b) wherein said controller accesses said patient-specific messages and said generic messages from said memory.

10. The memory treatment mechanism according to claim 9, wherein said controller selectively suspends the patient-specific messages from said subliminal content.

11. The memory treatment mechanism according to claim 10,
a) further including a switch selectively producing a signal; and,
b) wherein said control mechanism suspends patient-specific messages in response to the signal from said switch.

12. The memory treatment mechanism according to claim 11:
a) further including:
1) an electronic identifier worn by a human, and,
2) a sensor responsive to said electronic identifier and generating a presence signal when said electronic identifier is proximate to said sensor; and,
b) wherein said switch is responsive to said presence signal.

13. The memory treatment mechanism according to claim 12, wherein said sensor is positioned proximate to said television.

14. A memory treatment system comprising:
a) a controller creating at least two sets of electronic signals, each set of electronic signals having entertainment content and subliminal content, the subliminal content for each set of electronic signals being unique and containing patient-specific information; and,
b) at least two televisions, each of said televisions receiving one set of electronic signals.

15. The memory treatment system according to claim 14,
a) further including a memory containing at least two sets of patient-specific information; and,
b) wherein said controller accesses said at least two sets of patient-specific information from said memory.
